(19) United States
(12) Patent Application Publication
(10) Pub. No.: US 2026/0269047 A1
(43) Pub. Date: Sep. 10, 2026

(54) Profile Task Presentations, a New Value Profile Task, and Advanced Valuation Analysis

(71) Applicant: Robert Kinsel Smith, Dallas, TX (US)

(72) Inventor: Robert Kinsel Smith, Dallas, TX (US)

(21) Appl. No.: 19/071,119

(22) Filed: Mar. 5, 2025

(51) Int. Cl. G16H 20/70; G06Q 10/0639; G06Q 50/20

(52) U.S. Cl. CPC G16H 20/70; G06Q 10/06393; G06Q 50/2057

(57) ABSTRACT

This invention adds computations, analysis and reviews of valuations, and their relationships to one another, for a more precise modeling of how a user thinks and makes decisions. It provides significantly greater precision and applicability of value profiles by including analysis of all the different relationships that exist with corresponding pairs of valuations.

Secondly, this invention changes the methodologies behind the generation of the profile Tasks, making every user's profile Tasks different from those for every other user, and thus reducing the likelihood that a user will gain information from previous users that will enable that user to skew the results to desired outcomes.

Finally, this invention adds a third Task to the other two profile Tasks that has the user compare world and self value combinations, scores the user's comparisons, and tallies those scores to continually set ranges of significance.

### CROSS-REFERENCE TO RELATED PATENTS

This application is related to, and contains common disclosures with U.S. Pat. No. 6,338,628 B1, “PERSONAL TRAINING AND DEVELOPMENT DELIVERY SYSTEM”, and U.S. Pat. No. 6,618,723 B1, “INTERPERSONAL MOTIVATIONAL COMMUNICATIONS SYSTEM”, and U.S. patent pending: application Ser. No. 17/319,736, “ADVANCED VALUE PROFILE ANALYSIS DELIVERABLES AND APPLICATIONS” having the same inventor and owner, Dr. Robert Kinsel Smith. The related patents are hereby incorporated by reference into this description as fully as if here represented in full.

### BACKGROUND OF THE INVENTION

The present invention relates in general to computer-based training (CBT) and personal/professional development systems, and more particularly, to a personal tech device-implemented coaching, personal development, and advising system.

Since the first two patents were granted in the early 2000s employing formal axiology (value science), companies around the world have used the Clear Direction, Inc. products generated by the technologies of those two patents. The third patent application has been programmed into computer code and has been used solely by the author in his consulting business and his providing reports to ‘resellers’ to use in their consulting businesses. Now the marketplace's embracing and relying upon the Internet, smart phones, personal tech devices (smart watches, fit bits, ear buds, etc.), Apps, and Agents (Siri, Alexa, etc.) have both made more platforms possible and have made use of electronic devices common. Business professionals and consumers have grown to rely on these and readily embrace advances in their applications.

Secondly, the common perception concerning the shortening of the attention spans of Americans over recent decades is supported by the empirical evidence that people do not favor printed reports as they did previously and do not spend time or give much effort to learning things that are not immediately applicable.

Thirdly, video use on personal devices grew from 15% in 2013 to 66% in 2023 with countless sites, publications, and contests touting that videos ought to be only 7 seconds long. Between the observed changes and trends in consumers' activities and the demands we receive from clients, we have become convinced that resources that will be employed by today's adults are ones that provide immediate answers to present problems, brief high-action videos, advanced integration of machine learning and personal tech devices, and bullet-point advice and examples for personal and professional use and development.

For more than two decades Clear Direction, Inc. has provided printed reports explaining and detailing the results of clients' value profiles (the detailed process and description of these is found in the first patent application). The first reports were 155 printed pages in length. Because of client demands, in 2002 Dr. Smith reduced the size of the reports to 60 pages in length with 6 page electronic lessons (the first of his patents) sent to users via email over a six month period. With continual requests for greater simplicity, Dr. Smith developed a 19 page report in 2015, which has become Clear Direction's best-selling product.

Now clients are demanding even shorter ‘reports’ that are accessible electronically (for personal tech devices) and that employ the most advanced applications of technology (video, Agents, smart watches, etc.). This new level of brevity has required that the software precisely generate information from a user's profile results that heretofore has not been required. This level of precision from the scoring software has not previously been required because professional profile reviewers have interpreted, provided, and explained volumes of information to each user. Secondly, the interpretation algorithms (the scores and information are generated) used to date focus on the characteristics of each of the individual dimensions of value and only occasionally touch on how the clarities of those thinking dimensions gain their characteristics and meaning from how they interact and how they are inter-related. The comparisons heretofore have primarily only been with the user's rankings of the values with the axiological rankings, with the other comparisons noted in this application being overlooked and ignored.

Dr. Hartman, the founder of formal axiology, regularly emphasized that every profile ‘score’ was the result of how the user viewed the three dimensions in relation to one another. But the scoring and reports to date have emphasized each dimension's individual scores in relation to their axiological rank, marginally touching on any relationships to the other dimension capacities, and ignoring other important and relationships. This invention measures, reports, and prioritizes the inter-relatedness of each of the perspectives of the user from newly designed scoring algorithms and scores that represent and model the dynamic nature of the user's thinking. These are derived from and describe the dimension scores in light of their capacities and biases together, their relationships to the other dimensions, to the other field of reference, to the actions upon the values, and to user's differing views of the nature of values when acted upon constructively and destructively.

### A Brief Review of Formal Axiology

Formal axiology (value science) is a logical system that defines the terms of how people think and models how those terms interact and relate to one another for a person to make a decision. The terms define the different ways and properties of how a person can know something, another person or themselves. The modeling of those different terms employs transfinite calculus, a mathematical system that has sets with the same properties as the three dimensions of value. While different value scientists have argued that other mathematical models could be used, it is this author's conclusion that Dr. Hartman's use of transfinite set theory accurately models the logical system of value science and provides results that have proven to be highly reliable.

Since 1966 a value profile has been used to model a person's valuing (thinking, prioritizing, deciding). The resultant model is called a value structure. It provides scores on different number scales that represent the extent to which the subject valued each different dimension and how the subject related the different dimensions to one another.

Dr. Robert S. Hartman and others developed algorithms that led to models of how those different ways of thinking interact for each individual, based on their final rankings. Since the 1960s, individuals have completed a value profile and then received a personal report, accompanied by a professional's review of that report. The personal nature of the information along with the complexity of concepts made it difficult for a participant to get useful information from their reports without a professional's help. How a person values and how that applies to his/her life is full of complexity and is actually composed of ideas that are not commonly considered or discussed.

The value profile has been improved over the decades with the most culturally up-to-date and tested profile being the Hartman-Kinsel Profile (cf. U.S. Pat. No. 6,338,628 B1). All of Clear Direction, Inc.'s profile reports are generated from the results of the user completing the Hartman-Kinsel Profile. Back in the mid-1990's professional axiologists were expressing doubts as to whether a report could be constructed that did not require a professional's face-to-face interpretation. Professionals then concluded that most subjects' own ideas about themselves coupled to the difficulty of thinking about one's own thinking made it very difficult (if not impossible) to design a ‘report’ that would be informative and useful to these users.

In 2002 and 2003 Dr. Robert Smith was granted two patents for technologies employing formal axiology that generated useful reports. The first designed and generated ongoing lessons, based on each person's individual value structure, that addressed either their development as an employee or as a manager in business. The second patent provided personalized information to a manager on how best to lead his/her team, how he/her could manage more effectively in light of his/her own thinking biases, and how best to manage and communicate with each individual direct report. While these reports were not dependent upon professional reviews, they were still most useful when they were supplied with a professional review.

### The Value “Test”

As described in the first patent, each Task of the profile test contains 18 different phrases that represent one of 3 values to the power of one of 6 valuations (the noun is the value and the action upon or characteristic of the value is the valuation). The new art in this patent application improves the value “Tasks” and is described in detail below.

In order to rank these terms (that are formal representations of each of the value combinations) the user uses his/her ways of thinking to determine what those mean and how they compare to one another. Originally these tasks were completed with paper and pencil and by 1999 they were completed on computers connected to the internet. With the advancements in software and hardware, the options with which a user completes the value profile are greater, including personal computers and tablets, Agents, smart phones, and virtual headphones

Cognitive sciences tell us that these different dimensions of value are handled by different parts of the brain and therefore, like our senses, each person can have a different ‘formula’ of thinking depending on which brain regions have developed and are used. The output derived from the participant's ranking includes how that participant preferred the properties of certain dimensions over the properties of other dimensions, how consistently those preferences were displayed (versus significant reactions in specific cases), and how those preferences and consistencies relate to one another.

### Values and Valuations

In value science, the value is the noun and the valuation is the adjective or verb (qualifier) acting upon the noun. When the valuation is consistent with the properties and characteristics of the noun, then it adds value to that noun (like putting antifreeze in a car's radiator). When the action or adjective is inconsistent with the properties or characteristics of the noun, then it is a transposition, taking value away from that noun (like putting mud in a car radiator). The list of value combinations is 18 items, composed of three dimensions with three compositions on each and three transpositions on each, making a total of 18.

### A Brief Review of the Components of Formal Axiology

The following components are contained in the following algorithms (again explained in significant detail in the first patent application).

Dimensions of Value: In 1911, Dr. Bertrand Russell wrote of the three ways that humans know things: intrinsically (singularly), extrinsically (empirically), and systemically (conceptually). In the 1950s, Dr. Robert Hartman formally defined these three dimensions of value as:
Intrinsic: Singular value, being non-denumerable properties, coming from an infinite set of possibilities.
Extrinsic: Empirical value, being denumerable properties coming from an infinite set of possibilities
Systemic: Formal value, being denumerable properties coming from a finite set of possibilities.

Because Dr. Robert Hartman formalized the definitions of these terms, he was able to develop a formal system and the profile test, as described above. The first list on the test represents the world outside one's self (how we know things and other people). The common terms for some of this valuing include: empathy, tangible outcomes, logic, law, order, results, and sympathy. The second list on the test represented the three dimensions applied to one's self. Common terms for these kinds of knowing include: self-esteem, self-awareness, self-expectations, confidence, courage, resilience, and stubbornness. The new-art of this application is a third Task, comprise of both world and self value combinations.

Values are the nouns in the test's lists of statements. The three dimensions are identifiable by the elements in the definitions of the dimensions of value. Examples of these are: a person is intrinsic, a car is extrinsic, and a plan is systemic. In value science terms we would say that a person is intrinsic (singular), the person's body is extrinsic (empirical), and the person's ideas are systemic (formal constructs). Examples of values (nouns) in the tests include: baby, car, uniform, sign, ticket, mother, and food.
Valuations are the adjectives or actions which modify the values. Again these are definable in accordance with the definitions of the dimensions of value. Again, simple illustrations of these are: ‘life-saving’ being intrinsic, ‘useful’ being extrinsic, and ‘logical’ being systemic.

Compositions are valuations that are adjectives or actions acting upon the value (the noun) that increase the value of that noun. These are actions or properties that apply or enhance the noun. For example, a tablespoon of cream enhances a cup of coffee (for most people), so a cup of coffee with cream would be a composition.

Transpositions are valuations that are adjectives or actions upon the noun that reduce the value of the noun. These actions are ‘out of sync’ with the noun. An example of a transposition is ‘breaking a television with a hammer’. We can see that destroying a television may be a constructive action for the parent of a young child who wants to spend his entire day watching TV but in relation to the purpose and properties of televisions, breaking one with a hammer is an action that reduces that TV's value as a TV. Other transpositions include ‘hatred’ (intrinsic disvaluation), ‘destroying’ (extrinsic disvaluation), and ‘confusing’ (systemic disvaluation).

### ‘The Good’ and ‘Better’

Dr. Hartman identified that an axiom of value is the description of ‘the good’. That axiom is, “Something is good if it fulfills its definition.” A car is good if it is full of car properties while another car is fair if it has a few car properties. This definition led to Hartman defining ‘better’ as the thing that is ‘richer in properties’. This ‘richer’ is not a linear or arithmetic summing of the number of properties, but is that which has a greater combination of properties where their interaction results in that thing being richer or better than the others. The eighteen statements in a profile test (as listed above) are comprised of compositions and transpositions of each dimension with each type of valuation acting upon those. Because each dimension has a mathematical value (intrinsic=infinity to the infinite power, extrinsic=finite number to an infinite power, and systemic=finite number to a finite power), the different values with their valuations can be ranked numerically. This is called ‘the axiological rank’ with’ I to the power of I″ being the biggest number (infinity to the infinite power to the power of infinity to the infinite power) while ‘I sub I’ is the smallest number (infinity to the infinite power to the negative power of infinity to the infinite power). Hartman used the sets in transfinite set theory to position all of the combinations in relation to each other and then added more distinction by noting (in practice) that the infinity of the intrinsic dimension is greater than the infinity of the extrinsic dimension, and so forth. He listed this axiological ranking in his book, The Structure of Value and Dr. Smith furthered this work in ranking in his book (2007), Writing a Value Profile.

Dimensional Clarity is the consistency with the axiological rank of each of the nouns of the same dimension. When a participant's rankings of the six nouns in the test are consistently ranked in relation to the axiological rank, then the participant has demonstrated an ability to rank the statements without significant bias (not reacting or fixating on other aspects). When those rankings are far from their axiological position or some of them are ranked significantly differently from the other values of the same dimension, then the person's thinking about things of that dimension is unclear (due to a reaction or inability to see the values, valuations, or both). This can be likened to eyesight, where a person may be able to see clearly with one eye while being unable to see any detail with the other. Or it can be like being able to see great detail when objects are close and unable to see those same detail at all from a distance.

Thinking clarity is the ability to see and distinguish the elements of that dimension with clarity. People with unclear thinking are perceived to be stubborn, for they are unwilling to consider new ideas (data points) and apply them to their decision making. When a person has high thinking clarity, he is able to think about the new data, see how that new data does and does not apply, and is then able to integrate that data into a new idea, conclusion or decision.

### Computing the Profile Report (Dimensional) Scores

As outlined in the first patent, I combined the dims (the numeric scores representing the clarity of thinking for that particular dimension) and percent positive bias scores that Dr. Hartman developed to derive a profile report score (which I also call dimensional scores). This report score (introduced in the first patent) represents the combination of how the participant values things in that particular dimension (how biased toward or away from things in the dimension) with how consistently that person's rankings ranked in comparison to the axiological ranks for those value combinations. These report scores range from 1- 10. The matrix of scores used to assign these report scores is in the first patent. So the one number represented these two aspects of the person's value scores. I called these scores ‘profile report scores’. I created these because users found it very difficult to understand two different descriptors about each of the three world and three self dimensions. These profile report scores range from 1-10 with 6 being the center point of the scale. The closer the score is to six (5, 6, or 7), the clearer the thinking. Scores higher than 6 indicate a positive bias and lower than 6 a negative bias. So a score of 9 is a positive bias with poor clarity, and a score of 4 is a negative bias with a moderate clarity. A critical element in understanding the scoring is to remember that the biases and clarities arise from how the participant ranks the statements in relation to each other and then how those rankings compare to their position in the axiological rank.

### Explaining Each of the Six Individual Profile Scores

Historically the individual profile scores essentially stood on their own. An E2=8 was described according to what an 8 would indicate for extrinsic self valuing. The descriptions would not include how other thinking orientations may/could/do affect that thinking orientation.

Three other scores are historically presented in the Clear Direction reports. The first is the Types of Reasoning, which identifies if the participant ranked transpositions as compositions (and vis versa) and if so, how many times. Scores above 5 are fairly uncommon (fewer than 3% in the US population) and indicate thinking that is so out of the norm that those persons benefit by having their differences explained.

The second additional report score is Attention Balance. This is computed by comparing the scores and relationships of those scores to each other and to the axiological rankings to both the world thinking and self thinking. The outer ranges of this scoring scale indicate inner-directed (0.3) and outer-directed thinking (3), which are explained in the participant's report.

The third set of scores is an invention of Dr. Smith in the 1990s. It is derived from using the rankings of the valuations instead of the values in order to compute the scores. In other words, what looks like a score describing the user's valuing of things, other people and one's self, the score describes the user's valuing of different types of actions or descriptors (that are categorized by the defined nature of the three dimensions). Pertaining to objects and others, these can include adjectives like loving, useful, logical, irrational, personal, destructive, etc. Pertaining to one's self these can include confident, resilient, courageous, insecure, fearful, determined, persistent, insistent, etc. When these valuation biases are opposite the value biases for the same dimension, then those differences are reported and explained in the Clear Direction reports.

This application includes an improvement concerning the comparing each of the individual valuations in relation to the other valuations.

### A BRIEF SUMMARY OF THE INVENTION

While this is new art employing new phrases, creating new scores, and using new computer logic to score the relationships, I have chosen to call two of these ‘improvements’ because they are not new creations but improvements of what are commonly used by axiologists. Thus, I would say this invention is two new elements and one improvement heretofore absent from any applications of tools employing formal axiology:
Varied Tasks: an improvement of the value profile tasks that the contain value combination phrases that participants rank to derive their value structures. This improvement reduces the likelihood of ‘cheating’ by having the software randomly select one of many phrases from each group of representative phrases and position the selected ones in a random order. This way people cannot game the profile from others giving them clues on how to rank the statements.
An Additional Task: the inclusion of a third task to the present two tasks, from which an accurate description can be computed that identifies how the participant's world and self valuing compare to each other. This new task required new scoring algorithms and computer codes to be made useful for inclusion in the current report and advice.
Comparative Sums of Valuations: This application includes the creation of comparative sum scores and the scoring of the effects of valuations (both compositions and transpositions), enabling the comparing of each of those comparative sums with the other computed valuation comparative sums. These scores, that are processed by new code, will identify specific biases in the user's thinking when their valuation scores are significant.

Because it is this inventor's desire to maximize the benefits from a value profile, improving the precision of the reported results and measuring the participant's input concerning what here-to-fore has always been assumed, are paramount. With more reliable and precise content and continuing to utilize the advances in machine learning and agent technologies, it is our goal that our products be applied to solving problems heretofore out of the reach of value profile-based products.

The new task selection method and the additional task along with greater analysis of the composition and transposition relationships will enable users to improve their explanations, examples, counsel, and training for each individual participant. This invention makes it unlikely that participants will be able to benefit from others' experiences taking the profile (because each person will receive different phrases to rank). Additionally, this will provide the data the software will be able to analyze which, if any, phrases in the Tasks contain cultural biases that skew the computed results.

### The Basis of the Invention

The first element is making the user's ranking of the tasks different. This is done by making a group of different phrases that formally represent the same value combinations then having the computer randomly select a phrase from each group and randomly positioning each of those eighteen phrases for the participant to rank.
Up to this point, only one phrase representing each value combination has been used (for example “a good meal” is the only extrinsic valuation of an extrinsic value ever used in value profiles) and the order the value combinations has always remained the same (with E to the e always being the first phrase on the list of tasks).*{Special Note: While the Swedish Value Profile does not begin with an E to the e phrase, all of the phrases on their Tasks are in the same order and begin with the phrase that has the same value combination as their other Tasks.}
   This invention will be an improvement by making each valuing task different from every other task (mathematically there will be 1 in 1.124×10²⁸ possible combinations for each task). This will prevent users from benefitting from others' knowledge prior to their completing the profile Tasks themselves. This sharing has been a concern of consultants because on a number of occasions it has been discovered and had resulted in unreliable results. By way of history, if Dr. Hartman had this randomization feature, he would not have been conned by a con-artist who had memorized the order of the statements on the profile test. The con's perfect rankings caused Dr. Hartman to blindly trust him and lose the $10,000 he invested with this man.

The second element of this new art is the inclusion of a third task (with a new set of 18 phrases) for the participants to rank. Where the axiological tasks to date have only contained phrases that represent either the world/others-values or the self-values, this new list would include phrases of compositions of both the world-values and self-values. This new task will require that the participant value their own self in relation to the world (others). While consultants have talked about how their clients' views of the world differed from their views of themselves, this has never been measured directly. In his scoring manual, Dr. Hartman presented a set of scores that he titled the ‘balance quotients.’ These scores compared the overall judgment of the person's world and self thinkings, thus giving an indication of the overall judgment of world-thinking in relation to the self-thinking. These balance quotients accurately compare the participant's world and self dimensional judgments (their dims) but do not measure the person's biases.

While these scores provide very helpful information about stress tolerance and tendencies in communications, they do not provide a reliable comparison of the participant's self-views in relation to their world-views. This third task will have the participant directly rank world-value combinations in comparison to self-value combinations, thus providing how those compare in the mind of the participant.

The third element of this application takes the computation of each valuation (the dims of each value combination with its respective bias), determines an comparative sum of those computations of each category, and then compares those for each dimension of value (these value combination scores having been first included in the patent pending application (#17319736). The computing of the comparative sum of those valuations is for both compositions and transpositions, each of the three dimensions of value, and for the world and self valuations, totaling 12 different comparative sums. The new-art software code identifies when a particular comparative sum, representing an action upon a value, (whether a constructive-composition or a devaluing-transposition) is so significant in relation to all of the other comparative sums that it represents a dramatic and noteworthy thinking bias.

### Examples of the Three Elements of the New Art, Improving the Tasks

### Three Examples of the First Element:

Random listing of phrases to rank in the profile tasks that are randomly chosen from a group containing multiple phrases that represent the particular value combination:
Person A's Task A may begin with the following three phrases:
   A new car
   A mother's love for her newborn
   A train wreck
Person B's Task A may begin with the following three phrases:
   A confusing plan
   A deep passion for justice
   A house destroyed by fire
Person C's Task A may have the following first three phrases:
   A firefighter
   A life-saving medicine
   A traffic fine

### An Example of the Second Element:

A new list of eighteen statements (Task C) that are compositions of value applied to the world and applied to one's self:
A sample of 6 of the 18 statements in a Task C:
   A mother's love for her newborn
   Social norms are important to follow
   Doctors are smart
   I am good at what I do
   I love to be recognized for my skills
   My standards are good

### An Example of the Third Element:

Computing the comparative sums and determining significant valuation biases by summing the sums that pertain to the different categories (compositions/transpositions, the three dimensions of value, and the two frames of reference (world and self):
LOOKING AT INTRINSIC VALUATIONS (each dim with their respective valences)

Compare the sum of the two dimension scores with the sum of the four remaining scores and then take the absolute value of the difference for the final sum):

| | World | | | Self | | | |
|---|---|---|---|---|---|---|---|
| | I | E | S | I | E | S | Sums |
| Compos: | −1 | −2 | −5 | −2 | −2 | 2 | I: −3/−7 = 4 |
| | | | | | | | E: −4/−6 = 2 |
| | | | | | | | S: −3/−7 = 4 |
| Transpos: | −1 | −5 | 2 | 0 | −7 | 1 | I: −1/−9 = 8 |
| | | | | | | | E: −12/2 = 14 |
| | | | | | | | S: 3/−13 = 16 |
| Sums | −2/−10 = 8 | −7/−5 = 2 | −3/−9 = 6 | −2/−6 = 4 | −9/1 = 10 | 3/−9 = 12 | |

| | | |
|---|---|---|
| KEY SUMS: | Extrinsic C/T (14) | Systemic C/T (16) |

### What the New Art Provides:

1. An increased reliability that the participant did not get prior information from others about how best to rank the statements on the profile.
2. Information directly from the participants as to how their thinking about others (the world) differs in relation to their thinking about themselves.
3. A way to identify whether the participant has extreme biases about any of the valuation's actions upon the values. Such extremes would indicate a specific valuation bias that skews the person's overall decision making.

### THE KEY ELEMENTS OF THE INVENTION

1. The key elements for the improvement of the New Task Presentation is the library of multiple phrases that represent the designated value combinations and the code required to randomly select these and then randomly place them into a viable Task. One from each group will be selected and then listed in random order on the test. The library is:

### 1ARIIC:

A mother's love for her baby
A compassionate firefighter
A life dedicated to the life of another
A life filled with love
Sacrificing one's life for another person

### 2ARIiC:

I am a loving person
I deserve to be treated with dignity
I love who I am
I am a deeply passionate person
I am loved

### 1ARIeC:

A great athlete
The most powerful person
A friend
A well-known celebrity
Deep personal love that is demonstrated

### 2ARIeC:

I like who I am
I like myself, just as I am
I am a helpful person
I am an influencer
I am a person who makes a difference

### 1ARIsC:

A computer scientist
A smart advisor
Devotion to the truth
A passion for justice
A person who knows what is right

### 2ARIsC:

I am a smart person
I am a principled person
I am a truthful person
I am knowledgeable
I am 100% trustworthy

### 1AREiC:

A gift of love
Life saving medicine
Live saving deed
A successful heart transplant
A lovers' embrace

### 2AREIC:

What I do saves lives
I love to contribute
I love doing my best work
I love to win
I love what I do

### 1AREeC:

Winning an election
A bonus for doing good work
Helpful deeds
A reliable car
A new computer

### 2AREeC:

I am good at what I do
I like what I have done
My accomplishments show how capable I am
I like to win
I like to make things happen

### 1AREsC:

A traffic light
Doing what you promise
A well-designed work space
Giving correct directions
Following the instructions

### 2AREsC:

When I have a plan, I am happy
When I know what to do, I am effective
I am my best when I have a plan
I am most productive when I know what I should do
I follow my plans

### 1ARSIC:

A promise to love
Life-saving laws
Personal values
Information that saves lives
Creative ideas

### 2ARSIC:

My personal beliefs
I am passionate about what I believe
I love my commitments
I deeply care about what I believe
I am committed to being a loving person

### 1ARSeC:

A plan that works
A better idea
A motivating plan
Wise counsel
Laws that protect the environment

### 2ARSeC:

My accomplishments show what I believe
I perform according to my standards
My actions show my beliefs
My thoughts are very useful
My ideas are helpful

### 1ARSsC:

A logical idea
Understanding the rules
The rules of math
Computer languages
Legal citizenship

### 2ARSsC:

I know what I believe
I understand what I should do
My logic is consistent
I should obey my rules
I should meet my expectations

### 1ARIiT:

Using life to take a life
Hating another person
First-degree murder
A suicide bomber in a shopping mall
A serial killer

### 2ARIiT:

I hate myself
I hate people who love me
I have totally written off people who love me
I deeply wish I had not been born
I am unlovable

### 1ARIeT:

A person in a coma
A life lived in poverty
Poisoning a city's water supply
Using rockets to kill people
A person who tortures animals

### 2ARIeT:

I am a person who destroys things
I am useless
I am a person who no one wants
I have no life because I am not capable
What I have done has taken away my life

### 1ARIST:

An unjust person
An unfair referee
A racist
An unfaithful person
A liar

### 2ARIST:

I am a person who spreads falsehoods
I have no life when I think of my future
My life is lost because of my commitments
My faith is destroyed by what I have learned
There is no reason for me to continue to live

### 1AREIT:

Bombing a school full of people
A deadly drug overdose
Terminal cancer
Imprison an innocent person for life
An act of suicide

### 2AREiT:

I hate promoting my own skills and abilities
I hate what I do
I hate people who help me
I'm so depressed I can't do anything

### 1AREeT:

A flooded house
Polluted air
A car wrecked in an accident
Food poisoning
Poverty

### 2AREeT:

I don't like my job
I do not like my body
My accomplishments do not deserve recognition
What I do isn't contributing
My job is to prevent others from succeeding

### 1AREST:

Telling a lie
A speeding ticket
A foolish decision
Counterfeit money
Spreading fake news

### 2AREST:

What I do is stupid
My job is illogical
My efforts to help are inconsistent
I fail to succeed because I don't know what to do
I can't get things done because I don't know what is expected

### 1ARSiT:

Deadly promises
Plans to kill an individual
Ideas about killing
Prejudice
Revengeful thoughts

### 2ARSiT:

My feelings confuse me
I have hateful thoughts
I hate to be informed
My thoughts about my future cause me to feel lifeless
I hate my own standards

### 1ARSeT:

Goals that can't be reached
Unrealistic quotas
Unrealistic expectations
Bad ideas
An impossible promise

### 2ARSeT:

When I do my work, I get confused
I can't understand anything when I lose
I believe I should fail
My logic prevents me from doing my best
My thoughts go awry when I make a mistake

### 1ARSST:

Foolishness
Unfair policies
Legal loopholes
A confusing plan
A wrong answer

### 2ARSST:

I am confused about my responsibilities
I don't know what I should
I don't think I can meet my expectations
I am unsure about my future plans
I don't know what I believe

The logic and software code for this new art are presented in detail in the Detailed Description section.

2. The key elements of the New Task (Task C) are the phrases that represent the world and self value combinations, the logic and code required to score this new Task, and the software code to use machine learning to accurately update the range to determine whether scores are significant. As with the above described improvement of the Tasks, this Task would have its phrases selected and positioned randomly to be ranked by the participant. The software in the detailed description would generate scores that would key to the corresponding text files for the generating of the reports.

### New Task C

A life filled with love
When I have a plan, I am happy
I am passionate about what I believe
A passion for justice
A successful heart transplant
A reliable car
Rules of logic
I am a loving person
Life-saving laws
Wise counsel
I perform according to my standards
Computer languages
I am a person who makes a difference
A well-known celebrity
I am a principled person
I perform life-saving acts
My accomplishments show how capable I am
I understand what I should do

The logic and computer code for scoring and utilizing this Task is explained in detail beginning on page 30 of this application.

3. The Key Elements of the Advanced Valuation Analysis as new art includes the combining and scoring into relevant pairs of individual valuation scores (that were individually identified, labeled and computed in the previous patent application by this author) and comparing those pairs with the sums of the other four valuation scores. Comparing these pairs of valuations reveals biases in the user's thinking toward or away from the valuations represented by the measured pair. One of the elements of the previous patent application is the identifying and scoring of each individual composition and transposition for the world and self frames of reference. This improvement takes those valuations and combines them into pairs that relate to the different dimensions and frames of reference, which here-to-fore has not been studied or measured.

The computed valuation scores are used to create with new software, this new art:
1. the creation of a new score that assigns a bias value (+ or −) to each valuation along with its dim (it axiological accuracy) and
2. a methodology of summing pairs of these new scores to determine a total bias in those particular applications (dimensions of value, frames of reference, and composition/transpositions).
3. logic and code that automates the analysis of the effect of those computed scores on a user's overall decision making and puts that analysis into a form that enables the software code to convert those numbers into meaningful text for the different generated reports.

### DETAILED DESCRIPTION OF THE INVENTION

The new art and the improvements include software logic, algorithms, and newly developed and named scores that identify, measure, and direct the software to select what is reported in text (written or verbal) about the relationships of the elements of a person's value structure (the model of how a person values, derives meaning, prioritizes, and makes decisions). This invention introduces analysis of the compositions and transpositions that make up each individual profile score, scores for valuations that include the clarity and bias which affect the decision making of the participant, and the relationship of the value and valuations of the two frames of reference to one another.

As with the previous patents (both granted and pending for this author), this invention and improvements include the presented algorithms that direct the software to continually update the averages and standard deviations of the scores and relationships of each of the individual and relationship scores, keep those updates in a master database that includes the frequency of those scores, and analyze each user's profile scores in light of the axiological rankings and identifies patterns indicating scores closest to the axiological ranks and farthest away from the axiological rank. From this analysis, the software will prioritize the information in an ordered list for presentation to the user.

### Improvement #1: Improving the Presentation the Value Profile Tasks

This first element includes improving the methods that the tasks are created, thus eliminating the likelihood of a person cheating when preparing to take the profile. This new art requires the creation of multiple phrases with the same value combinations from which the software will randomly select for the current Task. This art requires software logic that will lead to a random selection from each group, placing the selected phrases in random order, and keeping track of where each phrase was placed in order to score the participant's answers in relation to the axiological ranks. These elements were not previously required because the same phrases were always used.

As with all Tasks, the final list will be comprised of 18 statements, representing intrinsic, extrinsic and systemic values being acted upon by compositions and transpositions of the same three dimensions. In this new art, those lists of phrases have almost no chance of ever being the same:
having different phrases representing each of the eighteen different value combinations and
having different positions on the list for the value combinations.
   The Computer Code for this Improvement:

This Python code, containing the different value combination phrases for Task A and Task B profiles in their defined sets, randomly selects one phrase from each of those 18 defined sets. It then places those 18 selected phrases for each Task in random order for the participant to rank best to worst.

```
Beginning of Code:
class PhraseOrderingApp(QWidget):
  def _{——}init_{——}(self):
    super( )._{——}init_{——}( )
    self.setWindowTitle(‘Phrase Ordering App’)
    self.layout = QVBoxLayout( )
    self.phrases = {
      “ARIiC”: [“A mother's love for her baby”, “A life filled with giving and
receiving love”, “A person who sacrifices her life for another person”, “A person who
loves everybody”],
      “ARIeC”: [“A well-known celebrity”, “A personal love that is shown by
actions”, “A life of usefulness and happiness”, “A passion to succeed”, “A good
doctor”, “A leader”, “A life full of successes”],
      “ARIsC”: [“A computer scientist”, “A licensed counselor”, “Devotion to the
truth”, “A life committed to justice”, “A person who knows what is right”, “An
adopted child”, “A genius”],
      “AREiC”: [“A gift of love”, “Life-saving medicine”, “Life-saving deed”, “A
successful heart transplant”, “Deeds of love”, “Technology that saves lives”],
      “AREeC”: [“Winning a contest”, “A bonus for doing good work”, “A
pollution-free factory”, “A technological advancement”, “Recognition for a good
deed”, “A strong and healthy body”, “A large donation to a charity”],
      “AREsC”: [“A traffic light”, “Doing what is planned”, “An efficient work-
space”, “A computer”, “Telling truthful news”, “Doing medical research studies”,
“Fulfilling a promise”, “A team uniform”],
      “ARSiC”: [“Lifesaving advice”, “A promise to love”, “Laws that save lives”,
“A family history”, “Personal values”, “Creative ideas”],
      “ARSeC”: [“A better idea”, “A plan that leads to victory”, “Helpful
information”, “A principle that motivates people to action”, “Laws that protect the
environment”, “Wise sayings”, “Scientific laws”],
      “ARSsC”: [“A logical idea”, “Understanding the rules”, “Fair policies”,
“Computer languages”, “Legal citizenship”],
      “ARIiT”: [“Personal hatred for another person”, “First-degree murder”,
“Faking love to commit murder”, “A person who loves to murder people”, “A life
with no love”, “A life consumed with hate”, “A terrorist”],
      “ARIeT”: [“A totally unproductive life”, “A life lived in poverty”, “Poisoning
a city's water supply”, “Killing people with drones”, “A person who tortures
animals”, “A person who likes to destroy”, “A life all alone, without friends”],
      “ARIsT”: [“A person who is not fair”, “An unfair referee”, “A racist”, “An
unprincipled person”, “An unfaithful person”, “A liar”],
      “AREiT”: [“Hatred of the natural world”, “Threatening to murder a
person's loved ones”, “Selling deadly drugs”, “Terminal cancer”, “Imprisoning an
innocent person for life”, “Selling a teenager into slavery”, “An act of suicide”],
      “AREeT”: [“A house flooded by a hurricane”, “A forest fire”, “Polluted air”,
“Hacking a smartphone”, “An injury from a fallen tree”, “Having your wallet
stolen”, “Poverty”, “Not being able to work”],
      “ARSiT”: [“Deadly promises”, “A plan to kill someone”, “A contract to kill”,
“Prejudice”, “Ideas that lead to hatred”, “Revengeful thoughts”, “A threat to
murder someone”],
      “ARSeT”: [“Goals that can't be reached”, “A traffic jam”, “Unrealistic
expectations”, “Impractical perspectives”, “Bad ideas”, “Unenforceable laws”, “An
impossible promise”],
      “ARSsT”: [“Foolishness”, “Unfair policies”, “A confusing instruction”, “A
wrong conclusion”, “A wrong answer”, “Nonsense”],
      “AREsT”: [“Telling fake news”, “An illegally parked car”, “An unjust
action”, “A foolish choice”, “Counterfeit money”, “An illegal vote”]
    }
    self.br_phrases = {
      “BRIiC”: [“I deserve to be treated with dignity”, “I love who I am”, “I am a
deeply passionate person”, “I am both loved and I am loving”],
      “BRIeC”: [“I like who I am”, “I like my life”, “I am a helpful person”, “I am
an influencer”, “I am a person who makes a difference”, “I am a good friend”],
      “BRIsC”: [“I am smart”, “I am a dependable person”, “I am truthful”, “I
am knowledgeable”, “I am totally trustworthy”, “I am a very loyal person”],
      “BREiC”: [“What I do saves lives”, “My work helps others feel loved”,
“When I do a good thing, I feel special”, “I do things out of my love for others”, “I
help others love each other”],
      “BREeC”: [“I am good at what I do”, “I like how I spend my time”, “My
accomplishments show how capable I am”, “I let my actions speak for me”, “I like to
win”, “I like to make things happen”],
      “BREsC”: [“When I have a plan, I am happy”, “I do good when I know what
I believe in”, “I am my best when I have a plan”, “I am most productive when I know
what I should do”, “I act faithfully to my principles”],
      “BRSiC”: [“I am passionate about what I believe”, “My promise is to love”,
“My values are who I am”, “I am committed to being a loving person”, “My
responsibilities are who I am”],
      “BRSeC”: [“My accomplishments show what I believe”, “I perform
according to my standards”, “My actions show my beliefs”, “My thoughts are very
useful”, “My knowledge is practical”, “My ideas are helpful”],
      “BRSsC”: [“I know what I believe”, “I understand what I should do”, “My
ideas are logical”, “My logic is consistent”, “I should obey my rules”, “I should meet
my expectations”],
      “BRIiT”: [“I have no imagination or passions”, “I hate people who love me”,
“I have totally written off people who love me”, “I deeply wish I had not been born”,
“I am unlovable”, “I feel invisible, as if I am not alive”],
      “BRIeT”: [“I am a person who destroys things”, “I have no life because of
things that were done to me”, “I am useless”, “I am a person who no one wants”],
      “BRIsT”: [“I am a person who spreads falsehoods”, “My ideas take my life
away from me”, “My life is lost because of my commitments”, “My faith is destroyed
by teachings I have learned”, “I cannot love because of how I think about things”,
“There is no reason for me to continue to live”],
      “BREiT”: [“I hate what I do”, “I hate people who help me”, “My present role
is destroying my life”, “I hate getting recognition or compliments”, “I don't feel I can
be useful”, “I'm so depressed I can't do anything”],
      “BREeT”: [“I don't like my job”, “I do not like my body”, “My body is not
attractive”, “My accomplishments do not deserve recognition”, “What I do is not
making a difference”, “My job is to stop others from succeeding”],
      “BREsT”: [“What I do is foolish”, “My job is illogical”, “My efforts to help
are unreliable”, “I fail to succeed because I am confused”, “I am ineffective when I
don't understand”, “I can't get things done because I don't know what is expected”],
      “BRSeT”: [“When I do my work, I get confused”, “I can't understand how to
change when I am losing”, “I believe I should not be better than others”, “I believe I
should fail”, “What I have done ruins my belief in myself”, “When I win I get
confused”],
      “BRSsT”: [“I am confused about my responsibilities”, “I don't know what I
should”, “I don't think I can meet my expectations”, “I am unsure about my future
plans”, “I don't know what I believe”]
    } # BR phrases dictionary
```

The following code randomly chooses a phrase that represents each value combination and puts it on the task page in random order. This is the code for Task B (the Self Task). Task A's code is identical with the “self” being replaced with “world”.

```
  def populate_phrases(self):
    self.selected_phrases = [ ]
    for key in self.M.keys( ):
      phrase = random.choice(self.phrases[key])
      self.selected_phrases.append(phrase)
    random.shuffle(self.selected_phrases)
    self.list_widget.clear( )
    for phrase in self.selected_phrases:
      item = QListWidgetItem(phrase)
      self.list_widget.addItem(item)
  def populate_br_phrases(self):
    self.selected_phrases = [ ]
    for key in self.br_phrases.keys( ):
      phrase = random.choice(self.br_phrases[key])
      self.selected_phrases.append(phrase)
    random.shuffle(self.selected_phrases)
    self.list_widget.clear( )
    for phrase in self.selected_phrases:
      item = QListWidgetItem(phrase)
      self.list_widget.addItem(item)
end of code
```

### New Art #2: Creation of a New Task

As noted in prior art, all of the dimensions of value and the terms in a value profile are formally defined. The relationships that are scored are logical interactions. So every score and relationship can be accurately described with words that correspond to the meanings of each of the scores and of the relationships.

The meanings and a sample of this procedure is upgraded in this new art by the addition of having the participants compare their valuing and valuations of others with their valuing and valuations of themselves. Over the decades a practical concern arose because axiologists noted that many participants commented about there being too many phrases to compare (clearly indicating that 18 phrases were a lot to compare).

This has led to a practical matter when designing a set of tasks to be ranked comparing world and self frames of reference. A complete set of phrases would include 36 different phrases (combining the already defined world and self phrases). 36 phrases would be untenable, so the questions arose whether only compositions should be listed (reducing the list to 18 phrases) or should the 24 extrinsic and systemic phrases be listed (with a emphasis on the nature of confidence and sources of direction that are contained in those two dimensions). This author concluded that the greater loss would come if the intrinsic comparisons were deleted (phrases which can provide valuable distinctions in light of the prevalence of narcissism and self-debasement that leads to suicidal behaviors). I concluded that the third Task would include phrases from all three dimensions in the two frames of reference with only the compositions being listed.

### The Elements of the Compositions of the Three Values in Two Frames of Reference

(key: 1 is world, 2 is self)

| | |
|---|---|
| 1I | A person, love, passion, life, personal feelings |
| 1E | Real empirical things, social, material success, power, |
| | energy, “want to” |
| 1S | Logic, ideas, formal constructs, order, ‘shoulds', |
| | principles, right/wrong |
| 2I | Myself, who I am, my inner being, my gestalt, my |
| | spiritual being, my life |
| 2E | What I do, how I appear, my initiative, confidence, body, |
| | how I compare |
| 2S | My principles, values, commitments, standards, goals, |
| | my self-definition |

### The Values of the Two Frames of Reference

| | |
|---|---|
| 1I | I value other's personal concerns, feelings, individuality |
| | more than I value myself, my being recognized/included, |
| | my own concerns/cares. |
| 2I | I value myself, my being recognized/included, my own |
| | concerns/cares more than I value other's personal concerns, |
| | feelings, individuality more than I value. |
| 1E | I value real empirical things, social, material success, |
| | power, energy, “want to” |
| 2E | I value what I do, how I appear, my initiative, confidence, |
| | body, how I compare, my actions within the empirical/social |
| | worlds I engage in, and my own benefits over yours. |
| 1S | I value and submit to logic, ideas, formal constructs, order, |
| | ‘shoulds', principles, right/wrong, expectations, and facts. |
| 2S | I am directed by my principles, values, commitments, standards, |
| | goals, and my own self-definition. |

### The Compositions for the Three Dimensions of Value Applying and to the Two Frames of Reference

| | |
|---|---|
| 1A Compositions | kind, attentive, caring, loving, honoring, |
| | personal |
| 1B Compositions | improving, making better, energetic, |
| | effective, useful |
| 1C Compositions | logical, reasonable, orderly, understandable, |
| | efficient, right |

Each frame of reference (world and self) includes intrinsic, extrinsic, and systemic values. The corresponding scores represent thinking about things and people with those types of properties. No one has developed a task that requires that the participant directly compare how s/he values others in comparison to how s/he values self. While professional reviewers have hypothesized about this comparison from how the individual world and self scores differ, those hypotheses are a stretch because those scores are derived from how the individual compared things within their own frames of reference (i.e. the intrinsic world score is derived from how the person compared world intrinsic values to world extrinsic and systemic values, not how the person compared world intrinsic values to self-intrinsic values).

In order to accurately measure the participant's perspectives on how these two frames of reference compare, the participant must actually compare them to each other. Dr. Hartman and my previous patent compare world and self thinking by comparing the dims (axiological accuracy). This comparison is accurate if the conclusion only notes how clearly the person can value the world in comparison to how clearly the person can value the self. Thinking clarity is measured by how close to the axiological rank a person's rankings are within the same frames of reference. Concluding how a person values the world in comparison to the self requires that the participant actually compare world and self values. This new art, where the participant does compare world and self values, will disclose the participant's predisposition for one of the frames of reference, while the axiological precision will continue to be derived from Tasks A and B.

As noted above, Task C will have 18 phrases that a participant ranks best to worst. Unlike the scoring of the dims and biases of Tasks A and B, the scoring for this will require new algorithms and codes.

This new art includes the following code to score Task C's world and self sums:

```
Beginning of code
TC = {
  “ARIi”: 1,
  “AREi”: 3,
  “ARSi”: 5,
  “ARIe”: 7,
  “ARIs”: 9,
  “AREe”: 11,
  “ARSe”: 13,
  “AREs”: 15,
  “ARSs”: 17,
  “BRIi”: 1,
  “BREi”: 3,
  “BRSi”: 5,
  “BRIe”: 7,
  “BRIs”: 9,
  “BREe”: 11,
  “BRSe”: 13,
  “BREs”: 15,
  “BRSs”: 17
}
PR_R_{——} = {
  “PR_Ii_1”: “A life filled with love”,
  “PR_Ei_3”: “A successful heart transplant”,
  “PR_Si_5”: “Life-saving laws”,
  “PR_Ie_7”: “A well-known celebrity”,
  “PR_Is_9”: “A passion for justice”,
  “PR_Ee_11”: “A reliable car”,
  “PR_Se_13”: “Wise counsel”,
  “PR_Es_15”: “A computer”,
  “PR_Ss_17”: “Rules of logic”,
  “PR_Ii_1”: “I am a loving person”,
  “PR_Ei_3”: “I perform life-saving acts”,
  “PR_Si_5”: “I am passionate about what I believe”,
  “PR_Ie_7”: “I am a person who makes a difference”,
  “PR_Is_9”: “I am a principled person”,
  “PR_Ee_11”: “My accomplishments show how capable I am”,
  “PR_Se_13”: “When I have a plan, I am happy”,
  “PR_Es_15”: “I perform according to my standards”,
  “PR_Ss_17”: “I understand what I should do”
}
WSR_{——} = {
  “WSR_Ii”: 0,
  “WSR_Ei”: 0,
  “WSR_Si”: 0,
  “WSR_Ie”: 0,
  “WSR_Is”: 0,
  “WSR_Ee”: 0,
  “WSR_Se”: 0,
  “WSR_Es”: 0,
  “WSR_Ss”: 0,
  “WSR_Ii”: 0,
  “WSR_Ei”: 0,
  “WSR_Si”: 0,
  “WSR_Ie”: 0,
  “WSR_Is”: 0,
  “WSR_Ee”: 0,
  “WSR_Se”: 0,
  “WSR_Es”: 0,
  “WSR_Ss”: 0
}
TotalWorldSum = 0
TotalSelfSum = 0
for key, value in WSR_{——}.items( ):
  if key.startswith(“WSR_”):
    term = key.replace(“WSR_”, “”)
    if term[0] == “I” or term[0] == “E” or term[0] == “S”:
      TotalWorldSum += value
    else:
      TotalSelfSum += value
print(“TotalWorldSum:”, TotalWorldSum)
print(“TotalSelfSum:”, TotalSelfSum)
end of code
```

The scores of the phrases (named according to their values and valuations) will follow a format from the software subtracting the user's rank from the axiological value (the code follows):

| | | | |
|---|---|---|---|
| Phrase | Axiological Value | User's Rank | Score |

| | | |
|---|---|---|
| 1Ii | 1 | 1IiSc = 1- UR1Ii |
| 2Ii | 1 | 2IiSc = 1- UR2Ii |
| 1Ei | 3 | 1EiSc = 3-Ur1Ei |
| 2Ei | 3 | 2EiSc = 3-Ur2Ei |
| 1Si | 5 | 1SiSc = 5-Ur1Si |
| 2Si | 5 | 2SiSc = 5-Ur2Si |
| 1Ie | 7 | 1IeSc = 7-Ur1Ie |
| 2Ie | 7 | 2IeSc = 7-Ur12e |
| 1Is | 9 | 1IsSc = 9-Ur1Is |
| 2Is | 9 | 2IsSc = 9-Ur12s |
| 1Ee | 11 | 1EeSc = 11-Ur1Ee |
| 2Ee | 11 | 2EeSc = 11-Ur2Ee |
| 1Se | 13 | 1SeSc = 13-Ur1Se |
| 2Se | 13 | 2SeSc = 13-Ur2Se |
| 1Es | 15 | 1EsSc = 15-Ur1Es |
| 2Es | 15 | 2EsSc = 15-Ur2Es |
| 1Ss | 17 | 1SsSc = 17-Ur1Ss |
| 2Ss | 17 | 2SsSc = 17-Ur2Ss |

The following three samples demonstrate why this is a valuable addition:

| | |
|---|---|
| World I to the e phrase's axiological rank is 4. | “Love of nature” |
| Self I to the e phrase's axiological rank is 4. | “I love what I do” |
| World I to the s phrase's axiological rank is 5. | “A mathematician” |
| Self I to the s phrase's axiological rank is 5. | “I am a principled |
| | person” |
| World E to the e phrase's axiological rank is 6. | “An effective medicine” |
| Self E to the e phrase's axiological rank ore | “I am good at what I do” |
| is 6. | |

We know that in the normal tasks, I to the e is 4th because it follows I to the i, E to the i, and S to the i. But now the two I to the e's are in places 7 and 8 because there are two of each pair prior to I to the e in this new task. The scoring also has to take into account that a rank of 7 or 8 are both axiologically correct because both 7 and 8 appropriately follow the first three phrases. Where Task C gets its value is in measuring how the person ranks the world phrases in comparison to the self phrases. So if the person is axiologically accurate in placing world I to the e as number 7 OR is axiologically accurate in placing the world I to the e as number 8, then whether it is 7 or 8 is demonstrating the bias toward the world or self and not demonstrating axiological accuracy.

The generation of this Task C will employ the same methods as the above presented improvement of the creation and presentation of Tasks A and B. Each Task C will be randomly selected from the multiple choices within the sets of phrases representing the same value and valuation and then randomly listed into Task C. Again, the likelihood of two Task Cs being identical is statistically impossible.

The scores for these samples are derived by subtracting the person's ranking of the phrase from the first of the two possible axiological positions. Those values will then be added and the world and self total values will be compared. The reason the ‘correct’ rank of 8 gets a value of −1, instead of 0, is because it has been ranked lower than its corresponding value combination from the other frame of reference. So, while it is accurately placed in relation to the other value combinations, it is lower than its corresponding value combination from the other frame of reference. When a totally different value combination jumps ahead of both of the subject value combination (say number 9 gets put in position 7), then the negative positions of the original 7s will reflect the positioning and that 9 combination will get a positive 2.

A sample of three different rankings of these six phrases:

| | Rank | Axiological Rank | Score (W/S) | |
|---|---|---|---|---|
| “Love of nature” | 7 | 7 | 0 | W |
| “I love what I do” | 8 | 7 | −1 | S |
| “I am a principled person” | 9 | 9 | 0 | S |
| “A mathematician” | 10 | 9 | −1 | W |
| “An effective medicine” | 11 | 11 | 0 | W |
| “I am good at what I do” | 12 | 11 | −1 | S |
| | | Totals: world = −1 self = −2 | | |
| “I love what I do” | 7 | 7 | 0 | S |
| “Love of nature” | 8 | 7 | −1 | W |
| “I am a principled person” | 9 | 9 | 0 | S |
| “A mathematician” | 10 | 9 | −1 | W |
| “I am good at what I do” | 11 | 11 | 0 | S |
| “An effective medicine” | 12 | 11 | −1 | W |
| | | Totals: world = −3 self = 0 | | |
| “Love of nature” | 7 | 7 | 0 | W |
| “A mathematician” | 8 | 9 | +1 | W |
| “An effective medicine” | 9 | 11 | +2 | W |
| “I am good at what I do” | 10 | 11 | −1 | S |
| “I am a principled person” | 11 | 9 | −2 | S |
| “I love what I do” | 12 | 7 | −5 | S |

Totals: world = 3 self = −8

Additionally, to the overall world/self comparison score, the individual IES sums will be computed, compared and converted into text assignments to provide meaningful information for the reports. This will follow the following logic:
Sum the scores (Tot=total) for the World and Self, I, E, and S values.

Code beginning
# Calculate the Total Scores
Tot1I = 1IiSc + 1IeSc + 1IsSc
Tot2I = 2IiSc + 2IeSc + 2IsSc
Tot1E = 1EiSc + 1EeSc + 1EsSc
Tot2E = 2EiSc + 2EeSc + 2EsSc
Tot1S = 1SiSc + 1SeSc + 1SsSc
Tot2S = 2SiSc + 2SeSc + 2SsSc
Code end

Compare the six Total Scores and identify those that are significant in comparison to the other five (either significantly more negative or positive). Significance will be continually evaluated, based on scores from ongoing profiles, but will start with scores being lesser than-8 or greater than 8.

```
Code beginning
# Compare the Total Scores
if Tot1I < −8 or Tot1I > 8:
  print(“Tot1I is a significant score.”)
if Tot2I < −8 or Tot2I > 8:
  print(“Tot2I is a significant score.”)
if Tot1E < −8 or Tot1E > 8:
  print(“Tot1E is a significant score.”)
if Tot2E < −8 or Tot2E > 8:
  print(“Tot2E is a significant score.”)
if Tot1S < −8 or Tot1S > 8:
  print(“Tot1S is a significant score.”)
if Tot2S < −8 or Tot2S > 8:
  print(“Tot2S is a significant score.”)
code end
```

Machine learning for the adjustment of the significant scores ranges as more profiles are processed. This keeps a running tally of the individual Tot scores and then adjusts range for setting the significance from 1 minus the average of the lowest Tot score to 1 plus the average of the highest Tot score:

```
Code beginning
# Update the running sum and count for each Total Score
# Initialize variables to store the running sum and count for each Total Score
Tot1I_sum = Tot1I
Tot1I_count = 1
Tot2I_sum = Tot2I
Tot2I_count = 1
Tot1E_sum = Tot1E
Tot1E_count = 1
Tot2E_sum = Tot2E
Tot2E_count = 1
Tot1S_sum = Tot1S
Tot1S_count = 1
Tot2S_sum = Tot2S
Tot2S_count = 1
# Initialize the significance ranges
lower_range = −8
upper_range = 8
while True:
  # Get the next set of Sc values
  new_1Ii, new_1Ie, new_1Is, new_2Ii, new_2Ie, new_2Is, new_1Ei, new_1Ee,
new_1Es, new_2Ei, new_2Ee, new_2Es, new_1Si, new_1Se, new_1Ss, new_2Si,
new_2Se, new_2Ss = get_next_sc_values( )
  # Calculate the new Total Scores
  new_Tot1I = new_1Ii + new_1Ie + new_1Is
  new_Tot2I = new_2Ii + new_2Ie + new_2Is
  new_Tot1E = new_1Ei + new_1Ee + new_1Es
  new_Tot2E = new_2Ei + new_2Ee + new_2Es
  new_Tot1S = new_1Si + new_1Se + new_1Ss
  new_Tot2S = new_2Si + new_2Se + new_2Ss
  # Update the running sum and count for each Total Score
  Tot1I_sum += new_Tot1I
  Tot1I_count += 1
  Tot2I_sum += new_Tot2I
  Tot2I_count += 1
  Tot1E_sum += new_Tot1E
  Tot1E_count += 1
  Tot2E_sum += new_Tot2E
  Tot2E_count += 1
  Tot1S_sum += new_Tot1S
  Tot1S_count += 1
  Tot2S_sum += new_Tot2S
  Tot2S_count += 1
  # Calculate the running average for each Total Score
  Tot1I_avg = Tot1I_sum / Tot1I_count
  Tot2I_avg = Tot2I_sum / Tot2I_count
  Tot1E_avg = Tot1E_sum / Tot1E_count
  Tot2E_avg = Tot2E_sum / Tot2E_count
  Tot1S_avg = Tot1S_sum / Tot1S_count
  Tot2S_avg = Tot2S_sum / Tot2S_count
  # Dynamically adjust the significance ranges
  lower_range = min(Tot1I_avg, Tot2I_avg, Tot1E_avg, Tot2E_avg, Tot1S_avg,
Tot2S_avg) − 1
  upper_range = max(Tot1I_avg, Tot2I_avg, Tot1E_avg, Tot2E_avg, Tot1S_avg,
Tot2S_avg) + 1
  # Compare the Total Scores to the dynamic significance ranges
  if Tot1I < lower_range or Tot1I > upper_range:
    print(“Tot1I is a significant score.”)
  if Tot2I < lower_range or Tot2I > upper_range:
    print(“Tot2I is a significant score.”)
  if Tot1E < lower_range or Tot1E > upper_range:
    print(“Tot1E is a significant score.”)
  if Tot2E < lower_range or Tot2E > upper_range:
    print(“Tot2E is a significant score.”)
  if Tot1S < lower_range or Tot1S > upper_range:
    print(“Tot1S is a significant score.”)
  if Tot2S < lower_range or Tot2S > upper_range:
    print(“Tot2S is a significant score.”)
  # Print the running sum and average for each Total Score
  print(f“Tot1I running sum: {Tot1I_sum}, running average: {Tot1I_avg}”)
  print(f“Tot2I running sum: {Tot2I_sum}, running average: {Tot2I_avg}”)
  print(f“Tot1E running sum: {Tot1E_sum}, running average: {Tot1E_avg}”)
  print(f“Tot2E running sum: {Tot2E_sum}, running average: {Tot2E_avg}”)
  print(f“Tot1S running sum: {Tot1S_sum}, running average: {Tot1S_avg}”)
  print(f“Tot2S running sum: {Tot2S_sum}, running average: {Tot2S_avg}”)
  print(f“Current significance ranges: lower = {lower_range}, upper = {upper_range}”)
end code
```

Why this is Important

An example of the importance of this measurement is found when two different users have the same extrinsic world score of 8 but having different world-self comparison scores (a Clear Direction report score of 8 indicates positive bias with some fuzziness in judgment/thinking clarity). While both persons are thinking similarly about extrinsic self-value as it compares to the intrinsic and systemic value about themselves (thus both having scores of 8), their application of their initiative, confidence, and desire to be seen as good may have totally different targets. The user with a self-valuing that is higher than his world-valuing will consider himself better (extrinsic) than others, will ‘need’ to win when competing, and will use his skills and efforts to feather his own nest/gain personal recognition. The user with a world-valuing that is higher than his self-valuing will apply his initiative, confidence, efforts and abilities to make the world better for others, help others, and contribute without needing to stand on a pedestal over his peers or colleagues. Both of these persons will initiate and both are confident in their ability to apply their extrinsic skills and energy while they have core differences in the desired outcomes from those efforts. The person with this 8 and a self-orientation will likely compete with his teammates, do what it takes to get ahead professionally, and potentially take credit for another's work. The other person will be confident and initiate for the good of others, make a positive difference, and will appreciate recognition, while not at the expense of others. Without this new art, these distinctions cannot be derived from an axiological test.

How this Information is Applied

Two alternatives will be added to all of the logic for the reports that are generated from this software. With the measurement of a person's bias toward or away from others, three different options exist when describing how the participant thinks and conclusions s/he will reach. The first is a strong positive bias favoring others. This can be in any of the three dimensions:
Intrinsic—others deserve to be treated with more dignity and compassion than I,
Extrinsic—others are more capable and deserving of recognition than I, and
Systemic—others are more reliable, loyal and logical than I.

A strong positive bias favoring one's self would result in the opposite conclusions.
Presently such biases are not reflected in the texts of the profile reports, so text that reflects these two extremes will be added. Alternative text fields will be added for when the applicable extreme is present and applies to what is being described. The logic will be:

### Beginning of Algorithm:

If TotalWorldSum+9>TotalSelfSum Then Go to E2DescriptionW, if not then go to E2DescriptionS if TotalSelfSum+9>TotalWorldSum, otherwise go to E2Description:
#Text if a strong bias toward others is computed:
   E2DescriptionW: “Your confidence and initiative drive you to want to be helpful, useful and make good things happen, even when the tasks are new to you.”
#Text if a strong bias toward one's self is computed:
   E2DescriptionS: “Your confidence and initiative drive you to want to succeed, win competitions, and get recognition for your efforts.′
#Text with no extreme scores:
   E2Description: “You have a good sense of initiative, confidence, and drive to have your efforts lead to desired outcomes.”
   End of algorithm

### a Detailed Description of #3, the Computing of Extreme Sums of Valuations

The resulting sums of the valuations are computed by adding the new scores of the specific composition or transposition scores from each frame of reference for each dimension of value and comparing that sum to the sum of the remaining four scores that are not in that set. The results are then subtracted from each other, giving a numeric value to the spread between the two sets (the final sum). The greater the spread, the greater the difference between the valuation being measured and the remaining valuations. If the spread is negative, then there is a significant bias against that particular valuation. If the spread is positive, then there is a significant bias for that particular valuation.
For example: as in the example on page 17, which is a chart for the actions upon Intrinsic Value, the systemic transpositions of the world and the self (1, 2) total 3 and the non-systemic transposition world and self scores (−7, 0, −5, −1) total −13. Subtracting −13 from 3 gives a sum of 16, which is a very significant positive difference (spread). This indicates that the systemic transpositions (illogical, unreliable, inconsistent, disorderly, etc.) are seen as being bad, while the intrinsic and extrinsic transpositions are not seen as being very bad. This person will naturally see and stand against the systemic transpositions being applied to individuals, while not seeing clearly and not really caring about intrinsic and extrinsic transpositions being applied to intrinsic values (persons, creativity, feelings, etc.).

The value of each individual composition's and transposition's computations were computed and stored in the software presented in the prior patent of this author (Application #17319736). These scores apply to Intrinsic, Extrinsic, and Systemic valuations, applied to Intrinsic, Extrinsic and Systemic values, which include Compositions and Transpositions, that are computed for the World and the Self frames of reference.

A spreadsheet with cells for each of the scores that are computed would be:
(First I, E, S are values, Second I, E, S, are valuations,
   W/S are world and self, C/T are compositions and transpositions)

### Intrinsic Valuations

| Values: | Intrinsic | Extrinsic | Systemic |
|---|---|---|---|
| World | | | |
| Compositions | IIWC | IEWC | ISWC |
| Transpositions | IIWT | IEWT | ISWT |
| Self | | | |
| Compositions | IISC | IESC | ISSC |
| Transpositions | IIST | IEST | ISST |

### Extrinsic Valuations

| Values: | Intrinsic | Extrinsic | Systemic |
|---|---|---|---|
| World | | | |
| Compositions | EIWC | EEWC | ESWC |
| Transpositions | EIWT | EEWT | ESWT |
| Self | | | |
| Compositions | EISC | EESC | ESSC |
| Transpositions | EIST | EEST | ESST |

### Systemic Valuations

| Values: | Intrinsic | Extrinsic | Systemic |
|---|---|---|---|
| World | | | |
| Compositions | SIWC | SEWC | SSWC |
| Transpositions | SIWT | SEWT | SSWT |
| Self | | | |
| Compositions | SISC | SESC | SSSC |
| Transpositions | SIST | SEST | SSST |

These thirty-six scores, being computed in the previous patent (pending), are all the individual scores computed from the two lists of eighteen phrases in Task A and Task B of the value profile. As presented earlier in this application, an example of this improvement is as follows:

### Computing the Final Sums

INTRINSIC VALUATIONS

| | World | | | Self | | | |
|---|---|---|---|---|---|---|---|
| | I | E | S | I | E | S | Sums |
| Compos: | −1 | | | −2 | | | I: −3/−7 = 4 |
| | | −2 | | | −2 | | E: −4/−6 = 2 |
| | | | −5 | | | 2 | S: −3/−7 = 4 |
| Transpos: | −1 | | | 0 | | | I: −1/−9 = 8 |
| | | −5 | | | −7 | | E: −12/2 = 14 |
| | | | 2 | | | 1 | S: 3/−13 = 16 |
| Sums | −2/−10 = 8 | −7/−5 = 2 | −3/−9 = 6 | −2/−6 = 4 | −9/1 = 10 | 3/−9 = 12 | |

Sums are computed by adding the two dimension scores in the different frames of reference (that we are identifying) and comparing that sum with the sum of the four remaining scores. In this example we are solving for the sum of I composition, so we add the world and self I scores in that row to each other (−1, −1) and then add the four non-I scores in that row to each other (−2, −5, −2, 2). The results are −3 and −7 respectively. Then taking the absolute value of the difference of those two sums, we get the final sum of 4. The final sums are then computed for each of the other rows and for each of the columns. The result is 12 final sums for Intrinsic valuations.

This computation is then done for the Extrinsic valuations and the Systemic valuations, resulting in 36 final sums that could be put into a spreadsheet like the one on the previous page.

Key sums are identified as those final sums that represent significant ‘distance’ between the particular sum and the sums to which it was compared. Final sums that are significant would be determined from analyzing hundreds of profiles with an initial indication that they would be displayed when their value is above the absolute value of 13.

| | |
|---|---|
| KEY FINAL SUMS: Extrinsic T (14) | Systemic T (16) |

The code to compute the above for INTRINSIC VALUATIONS is (the same code would be used for Extrinsic and Systemic valuations, based on those respective scores):

```
Beginning of code
intrinsic_valuations = {
  “Compos”: {
    “I”: {“World”: −1, “Self”: −2},
    “E”: {“World”: −2, “Self”: −2},
    “S”: {“World”: −5, “Self”: 2}
  },
  “Transpos”: {
    “I”: {“World”: −1, “Self”: 0},
    “E”: {“World”: −5, “Self”: −7},
    “S”: {“World”: 2, “Self”: 1}
  }
}
# Compute row sums
row_sums = {
  “Compos I”: abs((−1) + (−2) − (−2) − (−2) − (−5) + 2), # −3/−7 = 4
  “Compos E”: abs((−2) + (−2) − (−1) − (−5) + 2), # −4/−6 = 2
  “Compos S”: abs((−5) + 2 − (−1) − (−2) − (−2)), # −3/−7 = 4
  “Transpos I”: abs((−1) + 0 − (−5) − (−7)), # −1/−9 = 8
  “Transpos E”: abs((−5) + (−7) − (−1) − 2), # −12/2 = 14
  “Transpos S”: abs(2 + 1 − (−1) − (−5)) # 3/−13 = 16
}
# Compute column sums
column_sums = {
  “I”: abs((−1) + (−2) − (−1) + 0), # −2/−6 = 4
  “E”: abs((−2) + (−2) − (−5) − (−7)), # −7/−5 = 2
  “S”: abs((−5) + 2 − 2 + 1) # −3/−9 = 6
}
print(“INTRINSIC VALUATIONS”)
print(“ World Self”)
print(“ I E S I E S Sums”)
for frame in [“Compos”, “Transpos”]:
  print(f“{frame}:”.ljust(15), end=“”)
  for dimension in [“I”, “E”, “S”]:
    world_score = intrinsic_valuations[frame][dimension][“World”]
    self_score = intrinsic_valuations[frame][dimension][“Self”]
    print(f“{abs(world_score +
self_score)}/{sum([intrinsic_valuations[frame][d][‘World’] for d in
intrinsic_valuations[frame] if d != dimension] + [intrinsic_valuations[frame][d][‘Self’] for
d in intrinsic_valuations[frame] if d != dimension])}”.center(8), end=“”)
  print( )
print( )
print(“Final Sums:”)
for key, value in row_sums.items( ):
  print(f“{key}: {value}”)
for key, value in column_sums.items( ):
  print(f“{key}: {value}”)
# Print the final sums greater than 13
print(“\nSums Greater Than 13:”)
for key, value in row_sums.items( ):
  if value > 13:
    print(f“{key}: {value}”)
What is printed by this code:
Final results for INTRINSIC VALUATIONS
```

| | World | | | Self | | |
|---|---|---|---|---|---|---|
| | 3/7 | 2/7 | 5/9 | 2/4 | 9/1 | 3/−13 |
| Compos: | −1/−2 | −2/−5 | −5/2 | −2/−2 | −2/−2 | 2/7 |
| Transpos: | −1/−9 | −5/−12 | 2/3 | 0/−6 | −7/1 | 1/10 |

Final Sums:
Compos I: 4
Compos E: 2
Compos S: 4
Transpos I: 8
Transpos E: 14
Transpos S: 16
Sums Greater Than |13|:
Transpos E: 14
Transpos S: 16
end of code

### These Results Will be Used in Two Different Ways:

1. They will be called on by the software that generates reports for the participants for personal self-understanding and professional development. The statements will describe the participant's preference toward those valuations that garnered high sums in relation to the valuations to which it was compared.
   An example of such text:
      Your rankings indicate that you have a very strong aversion to others and yourself being disloyal, unreliable, unprincipled or confusing. The strength of these values comes as a result of these being of significantly higher value to you than other characteristics, like: unloving, mean, uncaring, impractical, useless, inflexible, and unchanging. Your strong preference can cause you to not be able to see the good when these other characteristics are present.
2. They will be an improvement (as noted with * in the list below), to the prioritization list (below: this is the list from the previous patent (applied), for the consultant's review. These valuation final sums are placed based on how high (very or extreme) each final sum is. The higher the final sum, the more that valuation plays a part in that participant's valuing and decision making.

### Improved: The Order of Prioritization for Profile Reviews

(*denotes new art of final sums)
1. Scores exactly on the axiological rank.
2. Scores close to the axiological rank and rarity.
3. Scores that are rare in comparison to other users.
4. Review of the participant's overall orientation (world/self/balanced).*
5. Significant patterns leading to strengths.
6. Elements and/or relationships that take command of all of the decisions (including values, extremely high {above 15} valuation final scores*, compositions, or transpositions).
7. Significant patterns between/among scores.
8. Significant outlier value, valuations (very high valuation final scores*), types of reasoning, and balance scores.
9. General descriptions of each of the six value scores including any disagreements between valuations, compositions, transpositions and corresponding values *.
10. Description of the interactions* in the Frame of Reference with the higher Rho.
11. Description of the interactions* in the Frame of Reference with the lower Rho.
12. Relationships that can undo strengths and result in misperceptions.

### #3 Detailed Description #3 Detailed Description #3 Detailed Description #3 Detailed Description

### EXAMPLES OF APPLICATIONS

The following list of applications is a summary of the applications in this author's prior patent application. The two improvements and the invention of the new Task do not change the possible applications, they improve the precision and reliability of the results, thus the applications remain the same.

In healthcare: equipping doctors and other healthcare providers to better communicate with their patients, in order to get more accurate information from the patients (the most common problem doctors report having is that their patients are not honest with them), more accurate ways to motivate and communicate expectations and instructions, and effective and technologically aided follow ups to increase the incidence of patient compliance and accuracy of taking prescribed medications.

In managing a business team: equipping the manager to more effectively communicate, direct, motivate, and assign tasks in light of that manager's own thinking and in light of each direct report's unique ways of thinking.

In building an effective team: providing the ability to assess the natural strengths and liabilities in a group of people that comprise a team. Different make ups are needed for teams to perform effectively at different tasks, within different markets, and under different conditions. Being able to evaluate what is needed (by using the categories of value science) a team head could quickly see if his/her team is comprised in a way that success is likely.

In providing constructive feedback: almost everyone is open to consider new ideas if they are approached in ways that fit their ways of thinking. This invention will provide a very precise roadmap that one could follow to increase the likelihood that feedback will be well received.

In psychology/therapy: being able to precisely identify the makeup of a person's low self-esteem, enabling the psychologist to target discussions and therapies that address the problem at the core, not at some general condition that may or may not respond to the therapist's remedies.

In coaching of athletes: distinguishing between general confidence, that causes an athlete to perform at higher levels when under pressure, and specific confidence that can cause an athlete to perform poorly under pressure.

In training and development: as the first patent outlined, the information from a value profile is very helpful in designing both an approach and a curriculum for training and development. This new invention, being significantly more robust and accurate, makes such training and development more precise and accurate.

Friendships, dating, marriage, parenting, and growing old: all benefit from the participants having accurate self-awareness. From not imposing one's own needs on the relationship, to being able to be engaged but not dependent, to being able to empathize and not sympathize, all the way to wanting to be in relationships while not needing to be in those relationships.

In career counseling and selection of a career track: The information gained from a value profile includes what motivates the user (and what demotivates that user), the kinds of interactions with others that are most enriching, the nature of the user's desire for practical outcomes at work, the importance of the mission of one's career or employer, and what secondary impediments could derail the user from his/her primary objectives.

In managing security personnel: Managers of police, security forces, and military forces would be able to identify potential problem personnel in their forces prior to significant problems occurring. Presently, such leaders are hesitant to dismiss or reassign personnel with one complaint for misconduct. The new art enables significant insight into the user's thinking, including thinking that supports prejudice, inability to function effectively under stress, and willingness and ability to follow orders when someone has broken the law.

What is claimed:

1. Inventing final sums for valuation pairs and the algorithms and code to generate the scores that quantify the differences of the pairs.

2. Designing and writing the algorithms and computer code to successfully score and identify valuation biases.

3. Utilizing the final sums to compare the valuations of each composition, transposition, of each dimension of value and of each frame of reference.

4. Producing differences of the final sums in relation to each other.

5. The development of the algorithms to accurately measure for and identify any strong biases in the user's thinking related to how values are acted upon, both world and self values (i.e. valuations).

6. The new art being the incorporating into reports based on the findings from users completing value profiles and being delivered, as claimed and awarded in previous patents of this user, in all manner of delivery: via the internet, computer sites, apps, personal technological devices including but not limited to smart phones, Ipads, smart watches, smart glasses, etc.

7. Designing the writing the logic and code that applies computed valuation biases to reports and instructions for consultants, that are previously described and outlined in this author's previous patents.

8. Providing the computing logic to continually compute and adjust the master list of the ranges of scores that constitute a user's extreme world/self bias.

9. Inventing the new Task and its components whereby the user compares world and self phrases.

10. The creation of the algorithms to create this new Task, and generating the code to compute, interpret, and present the user's overall bias toward or away from him/herself to the world and other persons.

11. The creation of machine learning code to continually adjust the lower and upper ranges of each dimensional total (world and self) to identify significant biases in the user's thinking.

12. Writing the algorithms that put the newly measured world/self scores into code that measures and identifies important distinctions and puts that information into useful forms for the information to be applied to the value profile reports.

13. Writing the algorithms and code that presents standard, value profile Tasks in formats (both the phrases themselves and the order in which the value combinations are listed) that vary from one user to the next.

14. Designing and writing multiple, axiologically-accurate phrases for each value combination, and writing the software that calls and employs those phrases in ways that generate profile Tasks that differ from one user to the next and are able to be scored as the Tasks have been in the past.

15. Providing the computing logic to continually compute and adjust the master list of the ranges of scores that constitute a user's extreme valuations biases.

16. Applying the newly gained information about the user's world and self biases to all of the products previously noted in the author's offerings, via any and all of the delivery options listed in previous patents and patent applications.
